(19) United States
(12) Patent Application Publication
(10) Pub. No.: US 2007/0203103 A1
(43) Pub. Date: Aug. 30, 2007

(54) Preparation of pharmaceutical salts of 3-O-(3',3'-dimethylsuccinyl)betulinic acid

(76) Inventors: Christian Hemp, Bacharach (DE); Arndt Hausherr, Mainz (DE); Theodore John Nitz, Boyds, MD (US); Roy Swaringen, Durham, NC (US)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 11/640,488

(22) Filed: Dec. 18, 2006

Related U.S. Application Data
(60) Provisional application No. 60/750,805, filed on Dec. 16, 2005.

(51) Int. Cl. A61K 31/56; C07J 53/00

(52) U.S. Cl. 514/169; 552/511

(57) ABSTRACT

This invention relates to a novel process for making 3-O-(3′,3′-dimethylsuccinyl)betulinic acid (“DSB”). This invention also relates to methods of treating HIV and related diseases using pharmaceutical compositions comprising salt forms of DSB prepared according to the process of the present invention. The invention further relates to dosage forms of pharmaceutical compositions comprising salts of DSB made using the process of this invention.

This application claims the benefit of priority of provisional U.S. Provisional Patent Application No. 60/750,805 filed 16 Dec. 2005, which is incorporated herein in its entirety.

### BACKGROUND OF THE INVENTION

1. Field of the Invention

This invention relates to novel processes for making 3-O-(3′,3′-dimethylsuccinyl)betulinic acid (“3-O-3′,3′-DSB”). This invention also relates to methods of treating HIV and related diseases using pharmaceutical compositions comprising 3-O-3′, 3′-DSB salt forms prepared according to the processes of the present invention. The invention further relates to dosage forms of pharmaceutical compositions comprising 3-O-3′,3′-DSB salts made using the processes of this invention.

2. Related Art

Human Immunodeficiency Virus (HIV) is a member of the lentiviruses, a subfamily of retroviruses. HIV infects and invades cells of the immune system; it breaks down the body's immune system and renders the patient susceptible to opportunistic infections and neoplasms. The immune defect appears to be progressive and irreversible, with a high mortality rate that approaches 100% over several years.

HIV-1 is trophic and cytopathic for T4 lymphocytes, cells of the immune system that express the cell surface differentiation antigen CD4, also known as OKT4, T4 and leu3. The viral tropism is due to the interactions between the viral envelope glycoprotein, gp120, and the cell-surface CD4 molecules (Dalgleish et al., Nature 312:763-767, 1984). These interactions, not only mediate the infection of susceptible cells by HIV, but are also responsible for the virus-induced fusion of infected and uninfected T cells. This cell fusion results in the formation of giant multinucleated syncytia, cell death, and progressive depletion of CD4 cells in AIDS patients. These events result in HIV-induced immunosuppression and its subsequent sequelae, opportunistic infections and neoplasms.

In addition to CD4+ T cells, the host range of HIV includes cells of the mononuclear phagocytic lineage (Dalgleish et al., supra), including blood monocytes, tissue macrophages, Langerhans cells of the skin and dendritic reticulum cells within lymph nodes. HIV is also neurotropic, capable of infecting monocytes and macrophages in the central nervous system causing severe neurologic damage. Macrophage/monocytes are a major reservoir of HIV. They can interact and fuse with CD4-bearing T cells, causing T cell depletion and thus contributing to the pathogenesis of AIDS.

Considerable progress has been made in the development of drugs for HIV-1 therapy. Therapeutic agents for HIV can include, but are not limited to, at least one of AZT, 3TC, ddC, d4T, ddI, tenofovir, abacavir, nevirapine, delavirdine, efavirenz, saquinavir, ritonavir, indinavir, nelfinavir, lopinavir, amprenavir and atazanavir, or any other antiretroviral drugs or antibodies in combination with each other, or associated with a biologically based therapeutic, such as, for example, gp41-derived peptides enfuvirtide (FUZEON; Trimeris-Roche), or soluble CD4, antibodies to CD4, and conjugates of CD4 or anti-CD4, or as additionally presented herein. Combinations of these drugs are particularly effective and can reduce levels of viral RNA to undetectable levels in the plasma and slow the development of viral resistance, with resulting improvements in patient health and life span.

Despite these advances, there are still problems with the currently available drug regimens. Many of the drugs exhibit severe toxicities, have other side-effects (e.g., fat redistribution) or require complicated dosing schedules that reduce compliance and thereby limit efficacy. Resistant strains of HIV often appear over extended periods of time even on combination therapy. The high cost of these drugs is also a limitation to their widespread use, especially outside of developed countries.

There is still a major need for the development of additional drugs to circumvent these issues. Ideally these would target different stages in the viral life cycle, adding to the armamentarium for combination therapy, and exhibit minimal toxicity, yet have lower manufacturing costs.

Betulinic acid derivatives, including 3-O-(3′,3′-dimethylglutaryl)betulinic acid and 3-O-(3′,3′-dimethylsuccinyl)betulinic acid, are known to have anti-HIV activity (U.S. Pat. No. 5,679,828). U.S. Pat. No. 5,679,828 mentions a synthesis that yields 70% 3-O-(3′,3′-dimethylsuccinyl)betulinic acid.

Kashiwada, Y., et al. (J. Med. Chem. 39:1016-1017 (1996)) mentions that the reaction between betulinic acid and 2,2-dimethylsuccinic anhydride in the presence of 4-(N,N-dimethylamino)pyridine and pyridine produces a mixture of two regioisomers: 3-O-(3′,3′-dimethylsuccinyl) betulinic acid (“3-O-3′,3′-DSB”) and 3-O-(2′,2′-dimethylsuccinyl) betulinic acid (“3-O-2′,2′-DSB”). Kashiwada et al. mentions that the EC₅₀ of 3-O-3′,3′-DSB is about four orders of magnitude lower than that of 3-O-2′,2′-DSB.

U.S. Pat. No. 6,172,110 mentions betulin derivatives comprising a 3-O-acyl and a(?) 28-O-acyl moeity.

U.S. patent application Ser. No. 10/870,555 (claiming priority to U.S. Provisional Patent Application No. 60/413,451 through U.S. patent application Ser. No. 10/670,797) mentions monoacylated betulinic acid derivatives.

Pokrovskii et al. mention that esterification of the 3-position carbon of betulin with succinic anhydride or a succinic acid derivative produces a compound capable of inhibiting HIV-1 activity (Pokrovskii, A. G. et al., Gos. Nauchnyi Tsentr Virusol. Biotekhnol. “Vector, ”9:485-491 (2001)).

U.S. patent application Ser. No. 11/081,802 mentions the N-methyl-D-glucamine and alkali metal salt forms of 3-O-3′,3′-DSB.

U.S. patent application Ser. No. 11/401,960 mentions crystalline polymorphs of N-methyl-D-glucamine (“NMG”) salts of 3-O-3′,3′-DSB.

Despite these advances, methods of making 3-O-3′,3′-DSB typically result in a mixture of starting material and the two regioisomers 3-O-3′,3′-DSB and 3-O-2′,2′-DSB. In some methods the regioisomeric purity of 3-O-3′,3′-DSB relative to 3-O-2′,2′-DSB is less than about 80%. For 3-O-3′,3′-DSB to be suitable for the medium or large scale manufacture of a pharmaceutical composition, there remains a long felt need for methods of synthesis that increase the regioisomeric yield of 3-O-3′,3′-DSB in relation to 3-O-2′,2′-DSB without additional purification steps. Thus, there remains a long felt need for a new process to make 3-O-3′,3′-DSB with regioisomeric purity of at least about 85%.

A process yielding 3-O-3′,3′-DSB with regioisomeric purity of at least about 85% relative to 3-O-2′,2′-DSB would satisfy a long felt need in pharmaceutical arts.

A process yielding 3-O-3′,3′-DSB with regioisomeric purity of at least about 90% relative to 3-O-2′,2′-DSB would satisfy a further long felt need in this art.

A process yielding 3-O-(dimethylsuccinyl)-betulinic acid with a purity of at least about 90% relative to the starting material betulinic acid would satisfy a further long felt need in this art.

A process yielding 3-O-(dimethylsuccinyl)-betulinic acid with a purity of at least about 95% relative to the starting material betulinic acid would satisfy a further long felt need in this art.

A process yielding 3-O-(dimethylsuccinyl)-betulinic acid with a purity of at least about 99% relative to the starting material betulinic acid would satisfy a further long felt need in this art.

### BRIEF SUMMARY OF THE INVENTION

One embodiment of the present invention relates to an improved process for preparing 3-O-3′,3′-DSB with a regioisomeric purity of at least about 85%. Another embodiment of the present invention relates to an improved process for preparing 3-O-3′,3′-DSB with a regioisomeric purity of at least about 90%. The process of the present invention comprises reacting DMSA with a salt of betulinic acid in the presence of a suitable solvent.

In another aspect, the present invention relates to a process yielding 3-O-(dimethylsuccinyl)-betulinic acid with a purity of at least about 90% relative to the starting material betulinic acid, such process comprising reacting DMSA with a salt of betulinic acid in the presence of a suitable solvent. Some such processes of the present invention, yield 3-O-(dimethylsuccinyl)-betulinic acid with a purity of at least about 95% relative to the starting material betulinic acid. Some such processes of the present invention, yield 3-O-(dimethylsuccinyl)-betulinic acid with a purity of at least about 99% relative to the starting material betulinic acid

In one aspect, the process of the present invention comprises preparing a salt of betulinic acid using a suitable base. In another aspect, the process of the present invention comprises preparing a mono- or di-cationic salt of betulinic acid by reacting a suitable mono- or di-cationic base with betulinic acid, e.g., by reacting betulinic acid with an equivalent amount or more of an alkali metal or alkaline earth metal hydride. Preferably, the alkali metal hydride is lithium hydride (LiH), sodium hydride (NaH) or potassium hydride (KH). More preferably, the alkali metal hydride is NaH. In some embodiments, the alkaline earth metal hydride may be calcium or magnesium hydride.

In another aspect, the process of the present invention comprises preparing an alkali metal or alkaline earth metal salt of betulinic acid by reacting betulinic acid with an equivalent amount or more of an alkali metal or alkaline earth metal alkoxide. In particular embodiments, the alkali metal or alkaline earth metal alkoxide may be lithium, sodium, potassium, magnesium or calcium alkoxide. In other embodiments, the alkali metal or alkaline earth metal alkoxide may be any linear or branched alkali metal or alkaline earth metal C₁-C₁₀ alkoxide, more preferably, any linear or branched lithium, sodium, potassium, magnesium or calcium C₁-C₁₀ alkoxide, more preferably, lithium, sodium or potassium C₁-C₄ alkoxides, even more preferably, lithium, sodium or potassium methoxide, ethoxide, or t-butoxide; most preferably, sodium methoxide.

In another aspect, the alkali metal or alkaline earth metal salt of betulinic acid is reacted with an excess of DMSA in a suitable solvent or a solvent mixture at about 60° C. to about 120° C. Tertiary amines are suitable solvents for the process of the present invention. Mixtures comprising tertiary amines may also be suitable solvents for the process of the present invention. Tertiary amines suitable for the process of the present invention include, but are not limited to, triethylamine, N,N-diisopropylethylamine, N,N-diisopropylmethylamine, N,N-di-n-propylethylamine, N,N-di-n-propylmethylamine, N,N-dimethylisopropylamine, N,N-dimethyl-n-propylamine, N,N-diethylisopropylamine, N,N-diethyl-n-propylamine, N,N-dimethybutylamine, N,N-dimethyl-sec-butylamine, N,N-dimethyl-tert-butylamine, N-methylpiperidine, N,N,N′,N′-tetramethylethylenediamine, N,N′-dimethylpiperazine, N-methylmorpholine, and combinations thereof. Some of the trialkylamines with boiling temperatures near or below room temperature (e.g. trimethylamine, N,N-dimethylethylamine and N,N′-diethylmethylamine) may become useful solvents for the process of the present invention if their volatility during the reaction is controlled by, for example, applying suitable pressure or mixing with a polar aprotic solvent. Other trialkylamines that are solid at or above room temperature may also be suitable for the process of the present invention if used as a mixture with a suitable polar aprotic solvent. For example, a sodium betulinate salt was reacted with an excess of DMSA in refluxing triethylamine. Other solvents that may be suitable for the process of the present invention are tetrahydrofuran, tetrahydropyran, 1,4-dioxane, dimethoxyethane, and combinations thereof. Those of skill in the art recognize that multi-solvent systems may be used where appropriate. This invention contemplates the use of multi-solvent systems.

In one aspect, DMSA is prepared by reacting 2,2-dimethylsuccinic acid with a slight excess of acetic anhydride. The acetic acid and the slight excess of acetic anhydride are removed from the product by co-distillation or azeotropic distillation.

### DETAILED DESCRIPTION OF THE INVENTION

The term “regioisomeric purity” as used herein means the relative amount or fraction of the desired regioisomer in the product mixture. Thus, a regioisomeric purity of 85% means the product mixture contains no more than 15% of all other undesired regioisomers.

The term “regioselective” or “regioselectively” as used herein means that a desired product, for example 3-O-3′,3′-DSB, is produced in greater yield than other possible products, for example 3-O-2′,2′-DSB.

The term “DABCO” as used herein means 1,4-diazabicyclo[2,2,2]octane.

The term “sodamide” as used herein means NaNH₂.

The term “LDA” as used herein means lithium diisopropyl amide.

The term “DBN” as used herein means diazabicyclononene.

The term “DBU” as used herein means diazabicycloundecene.

The term “DMSA” as used herein means 2,2-dimethylsuccinic anhydride.

The term “slight excess” as used herein in connection with reaction stoichiometry refers to the use of one equivalent of one reagent with from 1.05 to 1.15 equivalents of the second reagent. Thus, the second reagent is said to be in slight excess.

As used herein, the term “mono-cationic base” includes bases with a mono-cation, such as Li⁺, Na⁺, K⁺, tetraalkyl ammonium (e.g., N(CH₃)₄⁺) etc. NaH, KOC(CH₃)₃, LiN(CH(CH₃)₂)₂, (CH₃)₄NOH and NaOCH₃ are illustrative examples mono-cationic bases. The term “di-cationic base” includes bases with a di-cation, such Ca²⁺, Mg²⁺, Zn²⁺+, etc. CaH₂, Mg(OH)₂ and Zn(OCH₃)₂ are illustrative examples of di-cationic bases.

As used herein, the term “salt of betulinic acid” or “salts of betulinic acid” includes mono- and di-anionic salts, mono- and di-cationic salts, mixtures of salts, di-anionic salts with more than one type of cation and mono-anionic salts with more than one type of cation.

One aspect of the present invention is directed to a process of making 3-O-(3′,3′-dimethylsuccinyl)betulinic acid (“DSB” or “3-O-3′,3′-DSB”). 3-O-3′,3′-DSB is depicted in Formula I:

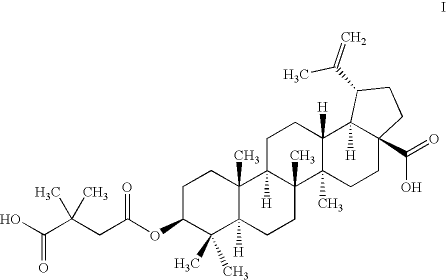

Another aspect of the present invention is directed to a process for preparing 3-O-3′,3′-DSB by providing a compound of Formula II:

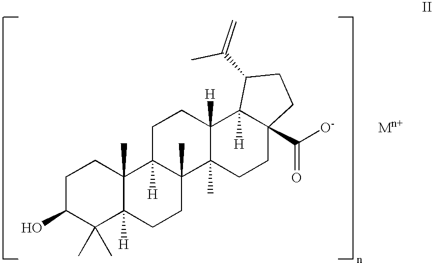

where Mⁿ⁺ is a cation with n oxidation state and n is 1 or 2,

and converting it to 3-O-3′,3′-DSB.

A further aspect of the present invention is directed to a process for preparing 3-O-3′,3′-DSB by providing a compound of Formula III:

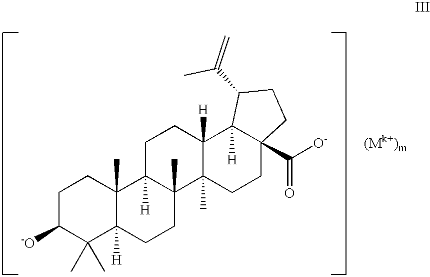

where M^{k+} is a cation with k oxidation state, and each of k and m is 1 or 2 provided that when m=1, k=2 and when m=2, k=1;

and converting it to 3-O-3′,3′-DSB.

A further aspect of the present invention is directed to a process for preparing 3-O-3′,3′-DSB by providing a compound of Formula IV:

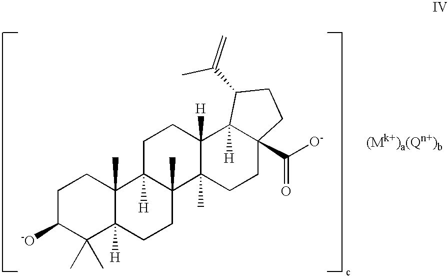

wherein each of M^{k+} and Qⁿ⁺ may be independently a mono- or a di-cation and therefore each of k and n may be independently 1 or 2; each of a and b may be between 0 and 2; and c may be either 1 or 2, provided that the electroneutrality of the salt is not violated. Mixed salts comprising a mono-cation and a di-cation, two different mono-cations, or two different di-cations may also be prepared and are contemplated by the present invention.

A further aspect of the present invention is directed to a glucamine salt of DSB, wherein DSB is made according to the process of the present invention. In one embodiment the glucamine salt of 3-O-3′,3′-DSB is the di-(N-methyl-D-glucamine) salt of DSB (3-O-3′,3′-DSB.2NMG). The di-(NMG) salt of 3-O-3′,3′-DSB has about two NMG molecules per 3-O-3′,3′-DSB molecule, a molecular formula of C₅₀H₉₀N₂O₁₆, a molecular weight of 975.28 and is depicted in Formula V:

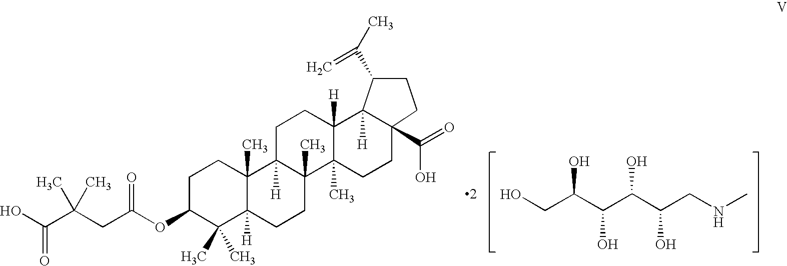

A further aspect of the present invention is directed to a pharmaceutical composition comprising 3-O-3′,3′-D.SB 2NMG made according to the process of the present invention, and a pharmaceutically acceptable excipient.

A further aspect of the present invention is directed to a dosage form, such as an oral tablet, comprising a pharmaceutical composition of an NMG salt of 3-O-3′,3′-DSB, in which the 3-O-3′,3′-DSB is made according to the process of the present invention. The dosage form can be used for treating a retroviral or lentiviral infection such as HIV in a subject.

A further aspect of the present invention is directed to a method of using a pharmaceutical composition comprising an NMG salt of 3-O-3′,3′-DSB, in which the 3-O-3′,3′-DSB is made according to the process of the present invention, for treating a retroviral or lentiviral infection such as HIV in a human subject.

In some embodiments of the present invention, the process comprises reacting betulinic acid with DMSA under conditions that favor the formation of 3-O-3′,3′-DSB over its regioisomer 3-O-(2′,2′-dimethylsuccinyl)betulinic acid (“3-O-2′,2′-DSB”) by a ratio of at least about 80:20, at least about 85:15, or at least about 90:10. Thus, reacting betulinic acid with an excess of DMSA in the presence of about one or more equivalents of a suitable base in a suitable solvent yields 3-O-3′,3′-DSB in high yield (e.g. greater than about 85% or greater than about 90%) and high regioisomeric purity (e.g. greater than about 85% or greater than about 90%). Solvents suitable for the process of the present invention are low-boiling tertiary amines. An example of such a solvent is triethylamine. Strong bases suitable for the process of the present invention include alkali metal alkoxides such as NaOCH₃ and alkali metal hydrides such as NaH. Suitable conditions for selectively synthesizing 3-O-3′,3′-DSB include heating the reaction mixture to about 50° C. to about 120° C., or to about 60° C. to about 100° C., or to about 70° C. to about 75° C.

In one aspect of the present invention, DMSA is first prepared by reacting 2,2-dimethylsuccinic acid with a slight excess, e.g., 1.05-1.15 equivalents, of acetic anhydride. The acetic acid and excess acetic anhydride are removed from the product by azeotropic distillation or co-distillation with toluene, yielding DMSA with a purity of at least about 97%. The present invention contemplates the use of multi-solvent systems where two or more solvents are present. In some embodiments, where the plurality of solvents form an azeotrope, azeotropic distillation is suitable. In some embodiments, where the plurality of solvents do not form an azeotrope, co-distillation is suitable. While certain examples in the present application may refer to either azeotropic distillation or co-distillation, one of ordinary skill in the art would recognize that changing one component of the multi-solvent system could require employing a different distillation technique.

Upon reaction of betulinic acid with an equivalent amount of a suitable base, a mono-anionic salt having Formula II is formed:

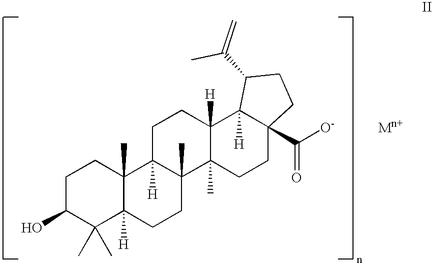

where Mⁿ⁺ is a cation with n oxidation state and n is 1 or 2. However, if betulinic acid is reacted with two or more equivalents of a suitable base, a di-anionic salt having Formula III is formed:

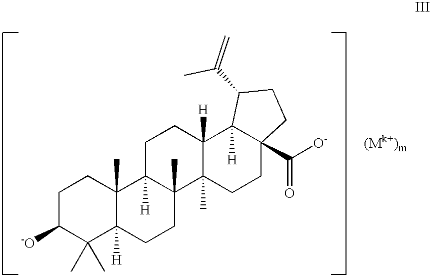

where M^{k+} is a cation with k oxidation state, and k and m may be 1 or 2 provided that when m=1, k=2 and when m=2, k=1. It will be recognized by those skilled in the art that mixtures of mono- and di-anionic salts will form when betulinic acid is reacted with between 1 and 2 equivalents of the base and that excess amount of base may be used.

In another embodiment of the present invention, bases may be used in combination with one another such that a salt with mixed cations or mixtures of salts, and combinations thereof, are prepared. To illustrate how a di-anionic salt with more than one cation is prepared, betulinic acid may be reacted with less than two equivalents of a first suitable mono- or di-cationic base in a first step, followed by less than two equivalents of a second suitable mono- or di-cationic base with a cation different from the cation used in the first step. It will be recognized that the amounts of each base can be varied (for example more than one equivalent of one base and less than one equivalent of the second base may be used), that mono-anionic salts of betulinic acid can be prepared by using one equivalent of the bases and that salts with more than two types of cations can be prepared by using more than two bases. For example, Formula IV allows for a mixed betulinic acid salt comprising a di-cation (e.g., M²⁺) and a mono-cation (e.g. Q¹⁺).]

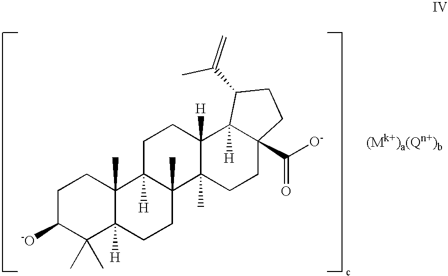

In Formula IV, each of M^{k+} and Qⁿ⁺ may independently be a mono- or a di-cation, each of k and n may be independently 1 or 2, each of a and b may be from 0 to 2, and c may be 1 or 2, provided that the electroneutrality of the salt is not violated. Mixed salts comprising two or more mono-cations, two or more di-cations and combinations thereof may also be prepared, for example (Al³⁺)₂ (3-O-3′,3′-DSB²⁻)₃ or (K⁺)(Na⁺)(3-O-3′,3′-DSB²−).

The skilled artisan will also recognize that the order of addition of the bases is not critical, that the bases can be mixed before reaction with betulinic acid and that the bases can simultaneously or sequentially be added into the reaction vessel. Similarly, salts of betulinic acid may be mixed prior to reacting them with DMSA.

Suitable alkali metal alkoxides for the process of the present invention include lithium, sodium and potassium alkoxide. Suitable alkali metal alkoxides include any linear or branched alkali metal C₁-C₁₀ alkoxide, preferably, any linear or branched lithium, sodium or potassium C₁-C₁₀ alkoxide, more preferably, lithium, sodium or potassium C₁-C₄ alkoxides, even more preferably, lithium, sodium or potassium methoxide, ethoxide or t-butoxide; most preferably, sodium methoxide.

Suitable alkali metal hydrides include lithium hydride (LiH), sodium hydride (NaH) and potassium hydride (KH). A preferable alkali metal hydride is NaH.

It will recognized by those skilled in the art that many other mono- and di-cationic bases are suitable for the preparation of betulinic acid salts, including bases with the following anions: amide (e.g., sodium amide); hydroxide (e.g., potassium hydroxide; tetramethylammonium hydroxide); carboxylate (e.g, sodium acetate and sodium pivalate); alkyl amide (e.g., sodium tert-butylamide), dialkylamide (e.g., lithium diisopropylamide); alkyl (e.g., n-butyllithium, sec-butylsodium, di-n-butylmagnesium); bis(trialkylsilyl)amide (e.g., potassium bis(trimethylsilyl)amide), and combinations thereof. Use of organometallic reagents comprising a reactive metal-carbon bond (e.g., Grignard reagents like methylmagnesium bromide) is also contemplated. Other organic bases suitable for the preparation of betulinic acid salts of the present invention include amidine bases (e.g., 1,8-diazabicyclo[5.4.0]undec-7-ene and 1,5-diazabicyclo[4.3.0]non-5-ene), guanidine bases (e.g., 7-methyl-1,5-7-triazabicyclo[4.4.0]dec-5-ene and N,N,N′,N″-pentamethylguanidine), and organic bases such as imidazole, 1-methylimidazole, 1,4-diazabicyclo[2.2.2]octane, tris[2-(2-methoxyethoxy)ethyl]amine, and combinations thereof. Inorganic bases that are suitable for the preparation of betulinic acid salts, include, but are not limited to, sodium carbonate, trisodium phosphate, and combinations thereof.

Those of skill in the art recognize that multi-base systems may be used where appropriate. This invention contemplates the use of multi-base systems. In some embodiments, processes of the present invention comprise the use of bases which are not-pharmaceutically acceptable, for example, in the preparation or purification of a pharmaceutically acceptable compound. All bases, whether pharmaceutically acceptable or not are included within the ambit of the present invention.

In some embodiments, the suitable base comprises an alkali metal base selected from the group consisting of sodium hydride, sodium hydroxide, sodium methoxide, sodium acetate, sodium pivalate, sodamide, trisodium phosphate, lithium hydride, n-butyl lithium, lithium methoxide, lithium diisopropyl amide, and potassium t-butoxide.

In some embodiments, the suitable base comprises an alkali metal base selected from the group consisting of sodium hydride, sodium methoxide, sodium acetate, sodium pivalate, sodamide, trisodium phosphate, n-butyl lithium, lithium diisopropyl amide, and potassium t-butoxide.

In some embodiments, the suitable base comprises an alkali metal comprising a sodium cation.

In some embodiments, the suitable base comprises an alkali metal comprising a lithium cation.

In some embodiments, the suitable base comprises an alkali metal comprising a potassium cation.

In some embodiments, the suitable base comprises an alkaline earth base.

In some embodiments, the suitable base comprises an alkaline earth base selected from the group consisting of calcium hydride, cesium hydroxide, magnesium hydroxide, magnesium methoxide, and methylmagnesium bromide.

In some embodiments, the suitable base comprises an alkaline earth base selected from the group consisting of calcium hydride, magnesium hydroxide, and magnesium methoxide.

In some embodiments, the suitable base comprises an organic base.

In some embodiments, the suitable base comprises an organic base selected from the group consisting of imidazole, DABCO, 1-methyl-imidazole, tris(methoxyethoxyethyl)amine, DBN, DBU, and 7-methyl-1,5,6-triazabicyclo[4.4.0]dec-5-ene.

In some embodiments, the suitable base comprises an organic base selected from the group consisting of imidazole, DABCO, 1-methyl-imidazole, and tris(methoxyethoxyethyl)amine.

In some embodiments, the suitable base comprises an alkali metal base selected from the group consisting of sodium hydride, sodium hydroxide, sodium methoxide, sodium acetate, sodium pivalate, sodamide, trisodium phosphate, lithium hydride, n-butyl lithium, lithium methoxide, lithium diisopropyl amide, potassium t-butoxide, imidazole, DABCO, 1-methyl-imidazole, tris(methoxyethoxyethyl)amine, DBN, DBU, and 7-methyl-1,5,6-triazabicyclo[4.4.0]dec-5-ene, calcium hydride, cesium hydroxide, magnesium hydroxide, magnesium methoxide, methylmagnesium bromide, and combinations thereof.

Suitable tertiary amines solvents include trialkylamines, triethylamine, N,N-diisopropylethylamine, N,N-diisopropylmethylamine, N,N-di-n-propyl ethylamine, N,N-di-n-propylmethylamine, N,N-dimethylisopropylamine, N,N-dimethyl-n-propylamine, N,N-diethylisopropylamine, N,N-diethyl-n-propylamine, N,N-dimethybutylamine, N,N-dimethyl-sec-butylamine, N,N-dimethyl-tert-butylamine, N-methylpiperidine, N,N,N′,N′-tetramethylethylenediamine, N,N′-dimethylpiperazine, N-methylmorpholine, and combinations thereof. In some embodiments, the solvent system is anhydrous. A trialkylamine with a boiling temperature near or below room temperature (e.g. trimethylamine, N,N-dimethylethylamine and N,N-diethylmethylamine) may become a suitable solvent if its volatility during the reaction is controlled by, for example, applying suitable pressure or if it is used as a mixture with a polar aprotic solvent. In some embodiments, the solvent system is heated to about 40, 45, 50, 55, 60, 65 or 70° C. A trialkylamine with a melting point at or above room temperature may become a suitable solvent is it is used as a mixture with a polar aprotic solvent. Other solvents that are suitable for the process of the present invention include tetrahydrofuran, tetrahydropyran, 1,4-dioxane, dimethoxyethane, and combinations thereof. Those of skill in the art recognize that multi-solvent systems may be used where appropriate. This invention contemplates the use of multi-solvent systems. Some embodiments employ binary solvent systems such as toluene and methylcyclohexane, toluene and ethanol, 2-butanol and methylcyclohexane, 2-butanol and ethanol, 2-butanol and acetonitrile, methanol and acetonitrile, or acetone and isopropylamine.

In some embodiments, an acid, for example, sulfuric acid, is used to initiate precipitation of 3-O-3′,3′-DSB from the solvent system.

In some embodiments, processes of the present invention are used to produce 3-O-3′,3′-DSB for the preparation of 3-O-3′,3′-DSB salts suitable for pharmaceutical compositions. Non-toxic, pharmaceutically acceptable amine or quaternary ammonium salts of 3-O-3′,3′-DSB would be suitable for pharmaceutical compositions. These salts can be prepared in situ during the final isolation and purification of 3-O-3′,3′-DSB or by separately reacting purified 3-O-3′,3′-DSB in its free acid form with a suitable organic base and isolating the salt thus formed. These include nontoxic ammonium, quaternary ammonium and amine cations including, but not limited to ammonium, tetramethylammonium, tetraethylammonium, and cations of methylamine, dimethylamine, ethylamine, N-methyl-D-glucamine and the like.

Of particular interest are the NMG salt forms of 3-O-3′,3′-DSB. One embodiment comprises 3-O-(3′,3′-dimethylsuccinyl)betulinic acid mono-N-methyl-D-glucamine. Another embodiment comprises 3-O-(3′,3′-dimethylsuccinyl)betulinic acid di-N-methyl-D-glucamine. Another embodiment comprises an alkali metal salt of 3-O-3′,3′-DSB. These salt forms are prepared by reacting 3-O-3′,3′-DSB with NMG or with an alkali metal hydroxide to provide mono- or di-salts of 3-O-3′,3′-DSB.

The 3-O-3′,3′-DSB salts prepared according to the process of the present invention have anti-retroviral activity, thus providing suitable compounds and compositions for treating retroviral infections, optionally with additional pharmaceutically active ingredients, such as anti-retroviral, anti-HIV, or immunostimulating compounds or antiviral antibodies or fragments thereof.

By the term “anti-retroviral activity” or “anti-HIV activity” is intended the ability to inhibit at least one of:

(1) viral pro-DNA integration into host cell genome;

(2) retroviral attachment to cells;

(3) viral entry into cells;

(4) cellular metabolism which permits viral replication;

(5) inhibition of intercellular spread of the virus;

(6) synthesis or cellular expression of viral antigens;

(7) viral budding or maturation;

(8) activity of virus-coded enzymes (such as reverse transcriptase, integrase and proteases); and

(9) any known retroviral or HIV pathogenic actions, such as, for example, immunosuppression. Thus, any activity which tends to inhibit any of these mechanisms is “anti-retroviral activity” or “anti-HIV activity.”

As used herein in connection with a measured quantity, “about” refers to the normal variation in that measured quantity, as expected by the skilled artisan making the measurement and exercising a level of care commensurate with the objective of measurement and the precision of the measuring equipment.

A 3-O-3′,3′-DSB salt of the present invention can be used for treatment of retroviral (e.g., HIV) infection either alone, or in combination with other modes of therapy known in the art. However, because the salts of 3-O-3′,3′-DSB of the present invention are relatively less or substantially non-toxic to normal cells, their utility is not limited to the treatment of established retroviral infections.

Pharmaceutical compositions of the present invention comprise at least one salt of 3-O-3′,3′-DSB prepared according to the process of the present invention, optionally in combination with one or more additional agents as described herein. Likewise, methods of treatment employ a pharmaceutical composition comprising at least one 3-O-3′,3′-DSB salt produced according to the present invention, as described herein, alone or in combination with additional agents as further described. Such modes of therapy can include chemotherapy with at least one additional drug as presented herein.

The terms “salt(s) of 3-O-3′,3′-DSB,” and “DSB salt(s) of the present invention” are used herein interchangeably and are intended to mean that the 3-O-3′,3′-DSB used in preparing the salt is made according to the process of the present invention.

In a preferred embodiment, products produced by the present invention are useful in the treatment of human patients.

The term “treating” means the administering to subjects a 3-O-3′,3′-DSB salt made according to the present invention for purposes which can include prevention, amelioration, or cure of a retroviral-related pathology.

Having now generally described the invention, the same will be more readily understood through reference to the following examples.

### EXAMPLE 1

A suspension of 219 g (1.5 mol) of 2,2-dimethylsuccinic acid in 220 L of toluene is heated to 130° C. (oil bath) and 168.4 g (1.65 mol, 1.10 eq.) of acetic anhydride is added drop-wise over 1.5 h. During the addition the suspension becomes a solution. After all the anhydride is added, the solution is stirred for further 1.5 h at 130° C. in oil bath. Toluene is removed by distillation. Addition of 250 mL of toluene and repeated distillation will remove residual acetic acid and acetic anhydride. Yield: 178 g (93%) slightly yellowish oil which crystallizes upon standing. Purity by NMR: 97%, traces of toluene, acetic acid, acetic anhydride

### EXAMPLE 2

A solution of 10 g (22 mmol) of betulinic acid in 42 mL of triethylamine preheated to 50° C. is added drop-wise to a suspension of 0.88 g (22 mmol) of NaH in 5 mL of triethylamine to form the sodium salt of 3-O-3′,3′-DSB. After addition is completed, triethylamine (23 mL) is distilled off, DMSA (7 g, 55 mmol) is added at 80° C. and the reaction mixture is refluxed. After 1.5 h HPLC indicates complete conversion and the suspension is cooled to 30° C. before it is carefully added to 75 mL of pre-cooled ethanol. Then, 45 mL of water followed by 30 mL of HCl (32%) are added and the product precipitates. The precipitate is filtered, washed with 100 mL of water and dried in vacuo at 50° C. overnight. Yield: 11.7 g (91%, crystals). HPLC: Betulinic acid: 0%; 3-O-3′,3′-DSB: 91.5%; 3-O-2′,2′-DSB: 8.5%.

### EXAMPLE 3

Betulinic acid (100 g, 219 mmol) is dissolved in 900 mL of triethylamine preheated to 50° C. Sodium methoxide (30% in methanol; 39 g, 215 mmol, 0.98 eq) is added and the glassware is rinsed with an additional 15 mL of methanol. The suspension is heated to 65° C. and 100 mL of a mixture of methanol/triethylamine is distilled off. The oil bath is then heated to 100° C. and an additional 400 mL of solvent are removed. 2,2-Dimethylsuccinic anhydride (70 g, 547 mmol, 2.5 eq.) is added and the suspension becomes a solution. An additional 140 mL of triethylamine are distilled off to obtain a ratio of 1:2.6 (betulinic acid:triethylamine). After 3 h, HPLC indicates complete conversion. Toluene (660 mL) is added and 600 mL of solvent (triethylamine and toluene) are removed by distillation. An additional 320 mL of toluene are then added and 320 mL of solvent are removed by distillation. The reaction mixture is cooled to 20° C., and HCl 37% (54 g, 547 mmol, 2.5 eq.) is added slowly followed by 200 mL of water. After the addition of 120 mL of methylcyclohexane, the mixture is stirred for 30 minutes at room temperature. The crystals are isolated, washed with 120 mL of toluene and 150 mL of water. The crude material is dried in vacuo at 50° C. overnight.

| | |
|---|---|
| Yield: | 109.5 g (89%) white powder. |
| HPLC: | Crystals: Betulinic acid: 0.15%; 3-O-3′,3′-DSB: 95.0%; |
| | 3-O-2′,2′-DSB: 4.4%. |
| | Mother liquor: Betulinic acid: 2.0%; 3-O-3′,3′-DSB: 69.5%; 3- |
| | O-2′,2′-DSB: 20.2%. |
| Assay: | 90% (unaccounted for residual organic solvents and water; no |
| | chloride contamination). |

### EXAMPLE 4

Sodium hydride (0.66 g, 60% in mineral oil, 16 mmol, 2.5 eq.) is added in portions to a solution of betulinic acid (3 g, 6.6 mmol) in triethylamine (10 mL). After stirring the suspension for 10 min, DMSA (1.7 g, 13.3 mmol) is added and the suspension is refluxed for 7 h. After 2 h the suspension thickens and an additional 20 mL of triethylamine are added.

After 7 h, HPLC indicates nearly complete conversion with the following regioisomeric selectivity: 3-O-3′,3′-DSB: 91%; 3-O-2′,2′-DSB: 8% (with 1% unreacted betulinic acid).

### EXAMPLE 5

Betulinic acid (1.4 g, 3.1 mmol) is suspended in 6 mL Et₃N, and heated to 50-55° C. under N₂. When the acid has been fully dissolved, the pale yellow solution is cooled to rt. Et₃N (0.75 mL) is added to the base (3.1 mmol) under N₂, and the solution of betulinic acid is added over 5 min. by syringe. The reaction mixture is stirred at rt for 30 min., and then heated to 70-75° C. At this temperature, 2,2-dimethyl succinic anhydride (0.99 g, 7.75 mmol) is added, and the reaction mixture is allowed to reflux under N₂.

Table 1 summarizes reactions performed in accordance with Example 5 comprising an organic base.

TABLE 1

| | Equivalents | Reaction | | Betulinic acid:3-O |
|---|---|---|---|---|
| Base | of Base | Time | | 2,2-DSB:3-O 3,3-DSB |
| DABCO | 1.0 | 24 | h | 0:15:85 |
| 1-methyl-imidazole | 1.0 | 24 | h | 0:17:83 |
| tris(methoxyethoxy- | 1.0 | 24 | h | 0:19:81 |
| ethyl)amine | | | | |
| DBN | 1.0 | 2 | h | 0:25:75 |
| DBU | 1.0 | 2 | h | 0:34:66 |
| 7-methyl-1,5,7- | 1.0 | 2 | h | 0:41:59 |
| triazabicyclo[4.4.0] | | | | |
| dec-5-ene | | | | |

Table 2 summarizes reactions performed in accordance with Example 5 comprising a metal-containing base.

TABLE 2

| | Equivalents of | Reaction | | Betulinic acid:3-O |
|---|---|---|---|---|
| Base | Base | Time | | 2,2-DSB:3-O 3,3-DSB |
| none (Et₃N | | 24 | h | 0:21:79 |
| present) | | | | |
| LDA | 1.0 | 48 | h | 2:12:86 |
| LiH | 1.0 | 6 | h | 1:17:82 |
| LiOCH₃ | 1.0 | 48 | h | 6:22:72 |
| NaH | 1.0 | 4 | h | 4:8:88 |
| Mg(OH)₂ | 0.5 | 24 | h | 0:18:82 |
| CH₃COONa | 1.0 | 24 | h | 0:20:80 |
| NaOH | 1.0 | 24 | h | 0:25:75 |
| KOₜ-Bu | 1.0 | 24 | h | 0:14:86 |
| CaH₂ | 0.5 | 24 | h | 0:18:82 |

### EXAMPLE 6

Betulinic acid is reacted with base as described in Example 5. After reaction with the base, 5 mL Et₃N is added, the reaction mixture is heated to reflux and 5 mL solvent is distilled off. This procedure is repeated two more times. Heat is then removed, 2,2-dimethyl succinic anhydride (0.99 g, 7.75 mmol) is added, and the reaction mixture is then allowed to reflux under N₂.

Table 3 summarizes reactions performed in accordance with Example 6 comprising a metal-containing base.

TABLE 3

| | Equivalents | Reaction | | Betulinic acid:3-O |
|---|---|---|---|---|
| Base | of Base | Time | | 2,2-DSB:3-O 3,3-DSB |
| CsOH | 1.0 | 48 | h | 6:26:68 |
| Mg(OMe)₂ | 0.5 | 4 | h | 0:7:93 |
| CH₃MgBr | 1.0 | 6 | h | 46:3:51 |
| CH₃MgBr | 2.0 | 6 | h | 75:1:24 |
| n-BuLi | 1.0 | 4 | h | 2:13:85 |
| NaOMe | 1.0 | 4 | h | 4:9:87 |
| (CH₃)₃CO | 1.0 | 4 | h | 0:10:90 |
| ONa | | | | |
| NaNH₂ | 1.0 | 24 | h | 0:10:90 |
| Na₃PO₄ | 0.33 | 24 | h | 0:10:90 |

Having now fully described the invention, it will be understood by those of ordinary skill in the art that the same can be performed within a wide and equivalent range of conditions, formulations and other parameters without affecting the scope of the invention or any embodiment thereof. All patents, patent applications and publications cited herein are fully incorporated by reference herein in their entirety.

What is claimed is:

1. A process for regioselectively preparing 3-O-(3′,3′-dimethylsuccinyl)betulinic acid in a greater yield than 3-O-(2′,2′-dimethylsuccinyl)betulinic acid comprising reacting 2,2-dimethylsuccinic anhydride with betulinic acid in the presence of a suitable base.

2. The process of claim 1, wherein the ratio of 3-O-(3′,3′-dimethylsuccinyl)betulinic acid to 3-O-(2′,2′-dimethylsuccinyl)betulinic acid is at least 80:20.

3. The process of claim 1, wherein the ratio of 3-O-(3′,3′-dimethylsuccinyl)betulinic acid to 3-O-(2′,2′-dimethylsuccinyl)betulinic acid is at least 85:15.

4. The process of claim 1, wherein the ratio of 3-O-(3′,3′-dimethylsuccinyl)betulinic acid to 3-O-(2′,2′-dimethylsuccinyl)betulinic acid is at least about 90:10.

5. A process for preparing 3-O-(3′,3′-dimethylsuccinyl)betulinic acid, comprising providing a compound of Formula II, III or IV: 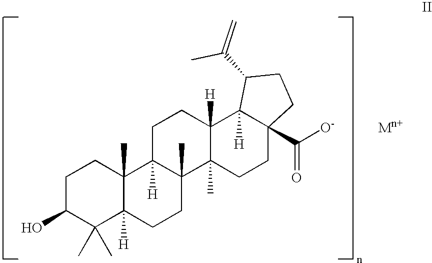
wherein Mⁿ⁺ is a cation with n oxidation states and n is 1 or 2; 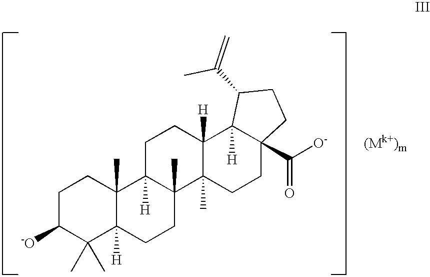
wherein M^{k+} is a cation with k oxidation state; each of k and m is 1 or 2 provided that when m=1, k=2 and when m=2, k=1; or 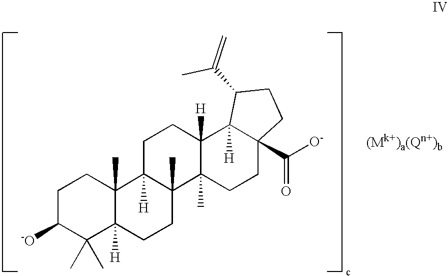
wherein each of M^{k+} and Qⁿ⁺ is independently a mono- or a di-cation, each of k and n is independently 1 or 2, each of a and b is from 0 to 2, and c is 1 or 2, provided that the electroneutrality of the salt is not violated;
and converting said compound to 3-O-(3′,3′-dimethylsuccinyl)betulinic acid.

6. The process of claim 5, wherein said compound is an alkali metal salt or an alkaline earth salt.

7. The process of claim 5, wherein said compound is a sodium salt.

8. The process of claim 5, wherein said converting said compound to 3-O-(3′,3′-dimethylsuccinyl)betulinic acid comprises contacting said compound with 2,2-dimethylsuccinic anhydride.

9. The process of claim 5, wherein said providing a compound of Formula II, III or IV comprises contacting betulinic acid with a suitable base.

10. The process of claim 9, wherein said suitable base is an alkali metal base.

11. The process of claim 10, wherein said alkali metal base is selected from the group consisting of sodium hydride, sodium hydroxide, sodium methoxide, sodium acetate, sodium pivalate, sodamide, trisodium phosphate, lithium hydride, n-butyl lithium, lithium methoxide, lithium diisopropyl amide, and potassium t-butoxide.

12. The process of claim 10, wherein said alkali metal base is selected from the group consisting of sodium hydride, sodium methoxide, sodium acetate, sodium pivalate, sodamide, trisodium phosphate, n-butyl lithium, lithium diisopropyl amide, and potassium t-butoxide.

13. The process of claim 10, wherein said alkali metal base comprises a sodium cation.

14. The process of claim 10, wherein said alkali metal base comprises a lithium cation.

15. The process of claim 10, wherein said alkali metal base comprises a potassium cation.

16. The process of claim 9, wherein said suitable base is an alkaline earth base.

17. The process of claim 16, wherein said alkaline earth base is selected from the group consisting of calcium hydride, cesium hydroxide, magnesium hydroxide, magnesium methoxide, and methylmagnesium bromide.

18. The process of claim 16, wherein said alkaline earth base is selected from the group consisting of calcium hydride, magnesium hydroxide, and magnesium methoxide.

19. The process of claim 9, wherein said suitable base is an organic base.

20. The process of claim 19, wherein said organic base is selected from the group consisting of imidazole, DABCO, 1-methyl-imidazole, tris(methoxyethoxyethyl)amine, DBN, DBU, and 7-methyl-1,5,6-triazabicyclo[4.4.0]dec-5-ene.

21. The process of claim 19, wherein said organic base is selected from the group consisting of imidazole, DABCO, 1-methyl-imidazole, and tris(methoxyethoxyethyl)amine.

22. The process of claim 5, further comprising the step of introducing a suitable solvent.

23. The process of claim 22 wherein the solvent is an amine.

24. The process of claim 23 wherein the solvent is a tertiary amine.

25. The process of claim 24 wherein tertiary amine is triethylamine.

26. The process of claim 5, wherein said providing a compound of Formula II, III or IV comprises contacting betulinic acid with an alkali metal hydride or an alkali metal alkoxide.

27. A process for preparing 3-O-(3′,3′-dimethylsuccinyl)betulinic acid, comprising:
(a) reacting a salt of betulinic acid with 2,2-dimethylsuccinic anhydride in a suitable solvent; and
(b) recovering the product.

28. The process of claim 27 wherein said salt is an alkali metal salt.

29. The process of claim 28, wherein said alkali metal salt is derived from an alkali metal base.

30. The process of claim 29, wherein said alkali metal base is selected from the group consisting of sodium hydride, sodium hydroxide, sodium methoxide, sodium acetate, sodium pivalate, sodamide, trisodium phosphate, lithium hydride, n-butyl lithium, lithium methoxide, lithium diisopropyl amide, and potassium t-butoxide.

31. The process of claim 29, wherein said alkali metal base is selected from the group consisting of sodium hydride, sodium methoxide, sodium acetate, sodium pivalate, sodamide, trisodium phosphate, n-butyl lithium, lithium diisopropyl amide, and potassium t-butoxide.

32. The process of claim 29, wherein said alkali metal base comprises a sodium cation.

33. The process of claim 29, wherein said alkali metal base comprises a lithium cation.

34. The process of claim 29, wherein said alkali metal base comprises a potassium cation.

35. The process of claim 27, wherein said salt is an alkaline earth metal salt.

36. The process of claim 35, wherein said alkaline earth metal salt is derived from an alkali metal base.

37. The process of claim 36, wherein said alkaline earth base is selected from the group consisting of calcium hydride, cesium hydroxide, magnesium hydroxide, magnesium methoxide, and methylmagnesium bromide.

38. The process of claim 36, wherein said alkaline earth base is selected from the group consisting of calcium hydride, magnesium hydroxide, and magnesium methoxide.

39. The process of claim 27, wherein said suitable base is an organic base.

40. The process of claim 39, wherein said organic base is selected from the group consisting of imidazole, DABCO, 1-methyl-imidazole, tris(methoxyethoxyethyl)amine, DBN, DBU, and 7-methyl-1,5,6-triazabicyclo[4.4.0]dec-5-ene.

41. The process of claim 39, wherein said organic base is selected from the group consisting of imidazole, DABCO, 1-methyl-imidazole, and tris(methoxyethoxyethyl)amine.

42. The process of claim 28, further comprising the step of introducing a suitable solvent.

43. The process of claim 42 wherein the solvent is an amine.

44. The process of claim 43 wherein the solvent is a tertiary amine.

45. The process of claim 44 wherein tertiary amine is triethylamine.

46. The process of claim 1, 5, 8 or 27, further comprising preparing 2,2-dimethylsuccinic anhydride by reacting 2,2-dimethylsuccinic acid with a slight excess of acetic anhydride and removing substantially all acetic acid from the reaction mixture.

47. 3-O-(3′,3′-Dimethylsuccinyl)betulinic acid having at least 85% regioisomeric purity relative to 3-O-(2′,2′-dimethylsuccinyl)betulinic acid prepared by a process consisting essentially of:
(a) reacting a salt of betulinic acid with 2,2-dimethylsuccinic anhydride in a suitable solvent; and
(b) recovering the product.

48. 3-O-(3′,3′-Dimethylsuccinyl)betulinic acid having at least 90% regioisomeric purity relative to 3-O-(2′,2′-dimethylsuccinyl)betulinic acid prepared by a process consisting essentially of:
(a) reacting a salt of betulinic acid with 2,2-dimethylsuccinic anhydride in a suitable solvent; and
(b) recovering the product.

49. The process of claim 1, 5, 12 or 22, further comprising contacting said 3-O-(3′,3′-dimethylsuccinyl)betulinic acid with N-methyl-D-glucamine.

50. An N-methyl-D-glucamine salt of 3-O-(3′,3′-dimethylsuccinyl)betulinic acid prepared according to the process of claim 49.

51. A salt of betulinic acid of the formula: 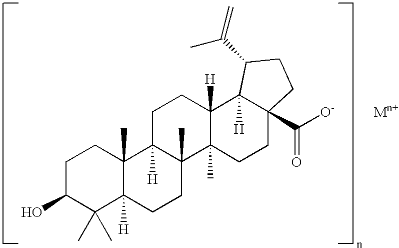
where Mⁿ⁺ is a cation with n oxidation state and n is 1 or 2.

52. A salt of betulinic acid of the formula: 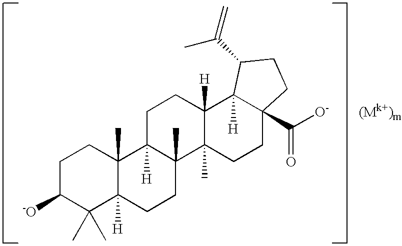
wherein M^{k+} is a cation with k oxidation state, and each of k and m is 1 or 2 provided that when m=1, k=2 and when m=2, k=1.

53. A salt of betulinic acid of the formula: 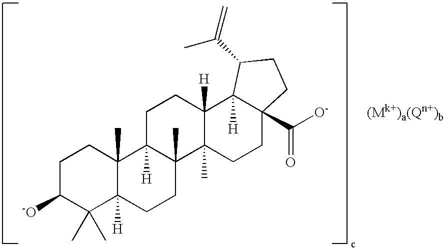
wherein each of M^{k+} and Qⁿ⁺ is independently a mono- or a di-cation, each of k and n is independently 1 or 2, each of a and b is between 0 and 2, and c is 1 or 2, provided that the electroneutrality of the salt is not violated.

54. A pharmaceutical composition comprising the salt of one of claim 49, 52 or 53, and a pharmaceutically acceptable excipient.

55. The pharmaceutical composition of claim 54, wherein the pharmaceutically acceptable excipient is a cyclodextrin.

56. A method of treating HIV infection comprising administering to a subject an effective amount of the pharmaceutical composition of claim 54.
